(19) United States
(12) Patent Application Publication
(10) Pub. No.: US 2025/0195822 A1
(43) Pub. Date: Jun. 19, 2025

(54) System and Method For Inducing Targeted Dreams Using Synchronized Sensory Cues and Sleep Phase Detection

(71) Applicants: Javier Agüera Reneses, New York, NY (US); Juliana Castro Varón, Brooklyn, NY (US)

(72) Inventors: Javier Agüera Reneses, New York, NY (US); Juliana Castro Varón, Brooklyn, NY (US)

(21) Appl. No.: 18/980,087

(22) Filed: Dec. 13, 2024

Related U.S. Application Data
(60) Provisional application No. 63/610,891, filed on Dec. 15, 2023.

(51) Int. Cl. A61M 21/00

(52) U.S. Cl. CPC A61M 21/00; A61M 2021/0016; A61M 2021/0027; A61M 2205/3303; A61M 2230/18

(57) ABSTRACT

A system and method for inducing memory-based dreams utilizes synchronized sensory cues and automated sleep phase detection. The system comprises a scent dispensing device with one or more chambers containing distinct scents, each associated with a unique identifier, and bio-sensors that detect user sleep parameters. A neural network processes the bio-sensor data to identify the N1 NREM sleep stage, triggering the coordinated delivery of olfactory and auditory cues associated with a selected memory. The system creates memory-sensory associations by linking specific scents with audio recordings and storing these relationships in a database. During the sleep cycle, embodiments monitor physiological parameters through various sensors, including wearable devices and smartphone sensors, to determine optimal timing for sensory cue delivery. Embodiments may incorporate machine learning algorithms to adapt and optimize cue timing based on user feedback and bio-sensor data, enhancing dream incubation effectiveness over time.

### FIELD OF THE DISCLOSURE

The present invention relates generally to systems and methods for influencing dream content through synchronized sensory stimulation, and more particularly to a computerized system that combines sleep phase detection, olfactory stimulus delivery, and audio playback to induce specific dream experiences. The invention further relates to the fields of neural network-based bio-signal processing, memory-sensory association systems, and adaptive learning algorithms for optimizing dream incubation effectiveness through multi-modal sensory coordination and physiological feedback analysis.

### BACKGROUND OF THE RELATED ART

Dream incubation, the practice of influencing dream content through pre-sleep stimuli, has historically been explored through various methodologies ranging from ancient cultural practices to modern scientific approaches. Early research in the 1960s and 1970s established fundamental connections between external stimuli during sleep and dream content, though these studies primarily relied on basic audio cues or visual stimulation without precise sleep phase targeting.

Recent technological developments in sleep science have enabled more sophisticated approaches to dream manipulation. The advent of consumer-grade electroencephalography (EEG) devices and smart wearables has made sleep stage detection more accessible outside laboratory settings. However, these technologies typically focus on sleep quality measurement rather than targeted dream manipulation, leaving significant potential in dream incubation applications unexplored.

Existing dream manipulation systems have predominantly relied on audio or visual stimuli delivered during REM sleep. While some success has been documented, these approaches often fail to leverage the powerful connection between olfactory stimuli and memory formation. Research has shown that the olfactory system maintains significant processing capabilities during sleep, with direct neural pathways to memory and emotion centers in the brain. Despite this understanding, practical applications combining olfactory and auditory stimuli for dream manipulation remain limited.

Current sleep monitoring systems typically employ single-modal sensing, such as actigraphy or heart rate monitoring, which can be unreliable for precise sleep stage detection. Multi-modal approaches incorporating various bio-sensors have shown promise in laboratory settings but have not been effectively translated to consumer applications. Additionally, existing systems often lack the ability to adapt to individual user responses and sleep patterns, resulting in sub-optimal stimuli timing and reduced effectiveness.

Traditional scent delivery systems have been primarily designed for ambient fragrance dispensing rather than precisely timed cognitive applications. While some programmable scent devices exist, they typically lack the temporal precision and integration capabilities necessary for effective dream manipulation. Furthermore, existing solutions have not adequately addressed the challenge of creating and maintaining consistent associations between specific memories and sensory cues.

The field of machine learning has made significant advances in processing complex bio-sensor data and identifying subtle patterns in physiological signals. However, these capabilities have not been fully utilized in the context of dream manipulation, particularly in combining multiple sensory modalities and optimizing stimuli delivery timing.

Prior attempts at memory enhancement during sleep have focused primarily on memory consolidation through audio replay of learning materials. While these approaches have shown some success in educational contexts, they have not fully explored the potential of combining multiple sensory modalities for more complex memory and dream manipulation objectives.

Some existing solutions have attempted to influence dreams through environmental control systems, manipulating factors such as temperature, lighting, and ambient sound. However, these approaches often lack the precision and personalization necessary for reliable dream incubation, and they fail to leverage the strong connection between specific memories and associated sensory cues.

The potential applications of effective dream manipulation extend beyond entertainment into therapeutic and educational domains. However, current solutions have not successfully integrated the various technological components necessary for reliable and personalized dream incubation. A system that could precisely coordinate multiple sensory stimuli with specific sleep phases while adapting to individual user responses would represent a significant advance in the field, enabling new applications in areas such as post-traumatic stress disorder treatment, creative problem solving, and memory enhancement.

### SUMMARY OF THE INVENTION

The following summary provides an overview of the memory-based dream incubation system and method of the present invention. This summary is not intended to identify key or essential elements of the invention or to delineate the full scope thereof. Rather, this summary presents certain concepts of the invention in a simplified form as a prelude to the more detailed description that follows.

In various embodiments, the invention provides a system for inducing memory-based dreams through coordinated delivery of sensory stimuli. The system may include a scent dispensing device that can accommodate one or more distinct scents, with each scent being digitally mapped to specific memories through unique identifiers. Sleep state detection may be accomplished through various bio-sensor configurations, including but not limited to wearable devices, smartphone sensors, or dedicated sleep monitoring equipment.

Aspects of the invention implement neural network architectures for processing bio-sensor data to identify optimal moments for sensory stimulation, particularly the N1 NREM sleep stage. These neural networks may be configured in various ways, such as using separate networks for different sensor types with a fusion layer, or employing a single end-to-end architecture. The system may utilize different machine learning approaches, including deep Q-learning or policy gradient methods, to optimize the timing and delivery of sensory cues.

Memory association functionality may be implemented through various user interfaces, allowing for the recording of audio cues and selection of corresponding scents. The system may communicate with peripheral devices through multiple protocols, such as BLUETOOTH, WI-FI, or Internet connections, providing flexibility in deployment configurations.

Some embodiments may incorporate feedback mechanisms that analyze both objective bio-sensor data and subjective user reports to enhance dream incubation effectiveness. The system may be implemented across different platforms, including mobile devices, cloud-based services, or standalone hardware configurations, with various options for data storage and processing architectures.

Embodiments also encompass methods for creating and managing memory-sensory associations, delivering synchronized sensory cues, and optimizing dream incubation parameters through machine learning techniques. These methods may be embodied in computer programs that coordinate the various system components and manage the dream incubation process.

### BRIEF DESCRIPTION OF THE DRAWINGS

The accompanying drawings illustrate several embodiments of the invention and, together with the description, serve to explain the principles of the invention according to the embodiments. One skilled in the art will recognize that the particular embodiments illustrated in the drawings are merely exemplary, and are not intended to limit the scope of the present invention.

FIG. 1 illustrates an exemplary memorable event showing a person witnessing a wedding ceremony taking place at a venue.

FIG. 2 illustrates another exemplary memorable event showing a person witnessing a child playing football at a venue with spectators.

FIG. 3 illustrates a person interacting with a scent canister that dispenses multiple distinct scents through individual chambers.

FIG. 4 illustrates a person using an audio recording device to capture audio cues for memory association.

FIG. 5 illustrates a memory creation input process showing the association between scent cue identifiers, audio cue identifiers, and resulting memory formation.

FIG. 6 illustrates a memory creation system showing the relationship between scent input, user data input, and memory storage in a database.

FIG. 7 illustrates a data coupling between a user device and scent canister, showing the interface for scent selection and control.

FIG. 8 illustrates a data communication system between a user device and a remote server through a communication network.

FIG. 9 illustrates a sleep environment setup showing a person sleeping on a bed with nearby user device, scent canister, and wearable device.

FIG. 10 illustrates a flow chart showing sensor data processing and trigger mechanisms for coordinated audio and scent cue delivery.

FIG. 11 illustrates the delivery of synchronized audio and scent cues to a sleeping person through integrated system components.

FIG. 12 illustrates a flow diagram showing the association, incubation, and reactivation phases of the memory-to-dream translation process.

FIG. 13 illustrates the implementation of machine learning algorithms for processing bio-sensor data and detecting sleep stages.

FIG. 14 illustrates the creation and management of crafted sensory cues including olfactory, auditory, and visual components.

FIG. 15 illustrates the system architecture showing the integration of hardware and processing components for dream incubation.

### DETAILED DESCRIPTION OF THE INVENTION

Unless otherwise defined, all technical terms used herein related to sleep science, neural networks, sensory stimulation, and dream manipulation have the same meaning as commonly understood by one of ordinary skill in the relevant arts of sleep technology, neuroscience, and cognitive science. It will be further understood that terms such as “sleep phases,” “neural networks,” “sensory cues,” and other technical terms commonly used in the fields of sleep science and dream research should be interpreted as having meanings consistent with their usage in the context of this specification and the current state of sleep monitoring technology. These terms should not be interpreted in an idealized or overly formal sense unless expressly defined herein. For brevity and clarity, well-known functions or constructions related to sleep monitoring systems, neural network architectures, or sensory stimulation mechanisms may not be described in detail.

The terminology used herein describes particular embodiments of the dream incubation system and is not intended to be limiting. As used herein, singular forms such as “a bio-sensor,” “a neural network,” and “the scent dispensing device” are intended to include plural forms as well, unless the context clearly indicates otherwise. Similarly, references to “sleep phase detection” or “sensory stimulation” should be understood to include multiple instances or variations of such elements, where applicable.

With reference to the use of the words “comprise” or “comprises” or “comprising” in describing the components, processes, or functionalities of the dream incubation system, and in the following claims, unless the context requires otherwise, these words are used on the basis and clear understanding that they are to be interpreted inclusively rather than exclusively. For example, when referring to “comprising a neural network,” the term should be understood to mean including but not limited to the described processing capabilities, and may include additional related functionalities or components not explicitly described. Each instance of these words is to be interpreted inclusively in construing the description and claims, particularly in relation to the modular and adaptive nature of the dream incubation system described herein.

Furthermore, terms such as “connected,” “coupled,” or “integrated with” as used in describing the interaction between various components of the system (such as between the bio-sensors and the neural network processor) should be interpreted to include both direct connections and indirect connections through one or more intermediary components, unless explicitly stated otherwise. References to “processing,” “detecting,” or “stimulating” should be understood to encompass both real-time operations and delayed or batch processing functionality, unless specifically limited to one or the other in the context.

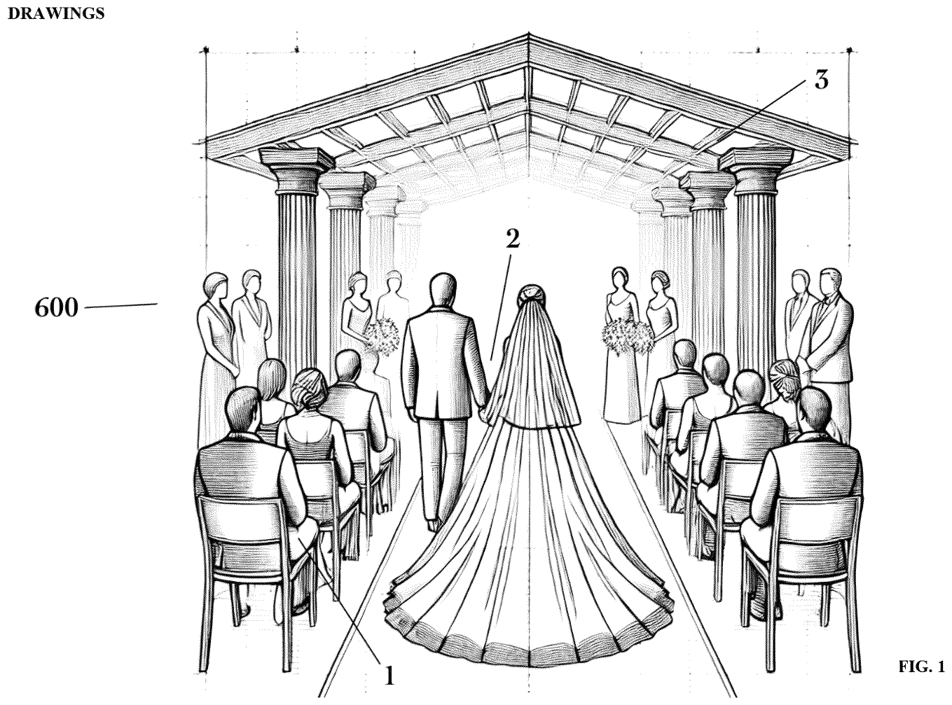

Referring now to FIG. 1, an exemplary memorable event is illustrated according to one aspect of the invention. The figure depicts a person 1 witnessing a first event 600, specifically a wedding ceremony, at a location or venue 3. In this embodiment, a couple 2 is shown performing their wedding ceremony. This represents one type of emotionally significant event that may be captured for dream incubation purposes. Weddings, being highly emotional experiences, typically engage multiple sensory pathways and create strong memory imprints, making them particularly suitable for the dream incubation system of the present invention. The emotional significance may stem from various aspects such as familial connections, romantic associations, cultural importance, or personal milestone recognition.

While FIG. 1 illustrates a wedding ceremony, other similar life milestone events may be equally suitable, including but not limited to graduation ceremonies, birth of a child, religious ceremonies, cultural celebrations, professional achievements, or personal accomplishments. The key aspect is the emotional resonance of the event with person 1, as stronger emotional connections typically lead to more vivid memory formation and subsequent dream experiences.

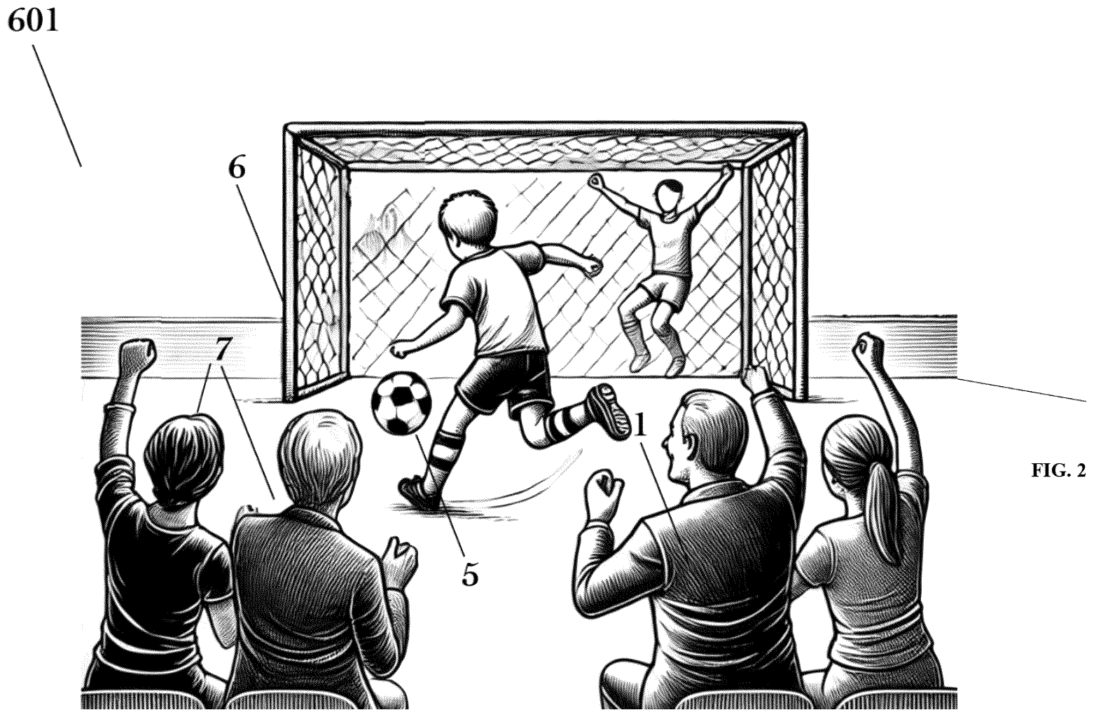

On the other hand, FIG. 2 illustrates another exemplary memorable event according to another aspect of the invention, where person 1 is witnessing a second event 601, in this case involving a child 4 playing with a football 5 at a second venue 6, with spectators 7 observing the football activity. This representation demonstrates how everyday moments may carry significant emotional weight, particularly when involving family members or personal achievements. The scenario might represent a child's first goal, a winning game moment, or simply a cherished parent-child interaction.

The invention contemplates various other types of memorable events suitable for dream incubation, such as travel experiences (e.g., first sight of a landmark, sunset over an ocean), personal accomplishments (e.g., winning a competition, completing a marathon), emotional encounters (e. g., reunions with loved ones, romantic moments), or adventure experiences (e. g., skydiving, mountain climbing). The common thread among these events is their capacity to create strong emotional impressions and engage multiple sensory pathways.

The emotional significance of the events depicted in FIGS. 1 and 2 may be amplified by various contextual factors such as the presence of loved ones, the achievement of long-held goals, or the culmination of significant effort or preparation. These emotional layers contribute to the formation of strong memory imprints that can be effectively targeted by the dream incubation system. The system may be particularly effective when the selected memories involve multiple sensory elements, such as distinctive sounds (wedding music, cheering crowds), visual elements (decorations, sports equipment), and associated scents (flowers, fresh-cut grass).

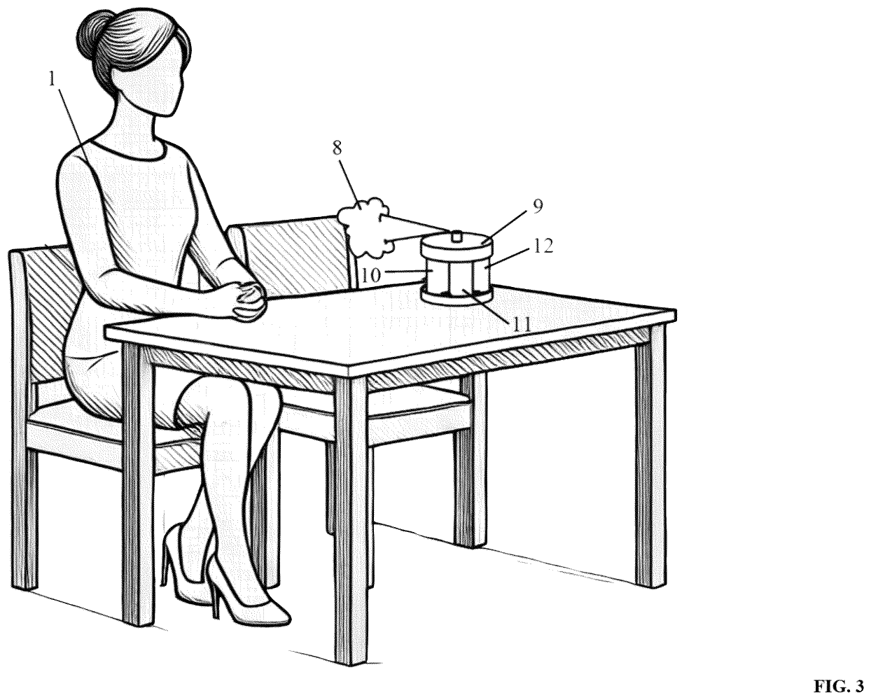

Referring now to FIG. 3, a person 1 is shown interacting with a scent canister 9 according to one aspect of the invention. The scent canister 9 may be configured to dispense scent fumes 8 comprising multiple distinct scents 10, 11, and 12. The scent canister 9 may be implemented in various forms, including but not limited to a programmable electronic scent diffuser, a multi-chamber dispensing device, a single-chamber dispensing device, or a smart home fragrance system. Each chamber within a canister may be individually addressable through electronic controls, allowing for precise selection and dispensation of specific scents. Such devices are well known in the art.

The scent canister 9 may incorporate various dispensing mechanisms such as piezoelectric atomization, heated evaporation, or ultrasonic diffusion. In some embodiments, the canister may include multiple separate pods or cartridges, each containing a different scent and associated with a unique digital identifier in the system. Alternative implementations may include networked arrays of single-scent dispensers, smart potpourri devices, or electronically-controlled essential oil diffusers.

Further, the scent canister 9 shown in FIG. 3 may preferably incorporate a wireless communication system enabling remote operation through BLUETOOTH™ and WI-FI™ protocols. The canister may include an integrated controller that manages scent selection and dispensation from the multiple chambers containing scents 10, 11, and 12. The canister may include only one chamber. The controller may preferably process commands received wirelessly from user devices or central servers to activate specific scent chambers and control dispensation parameters.

The canister may be implemented as a rechargeable device incorporating a lithium-ion battery system, enabling portable operation while maintaining consistent scent delivery capabilities. The battery capacity may preferably support multiple nights of operation, with charging accomplished through standard USB interfaces or wireless charging systems. The controller may implement power management protocols to optimize battery life, adjusting dispensation mechanisms and wireless communication parameters based on battery status.

The canister's wireless capabilities may enable integration with home automation systems while maintaining the precise temporal control required for dream incubation. Battery status monitoring and low-power alerts may be transmitted to connected devices through the established wireless connections, ensuring reliable system operation. Such scent canister implementations are equally well known in the art.

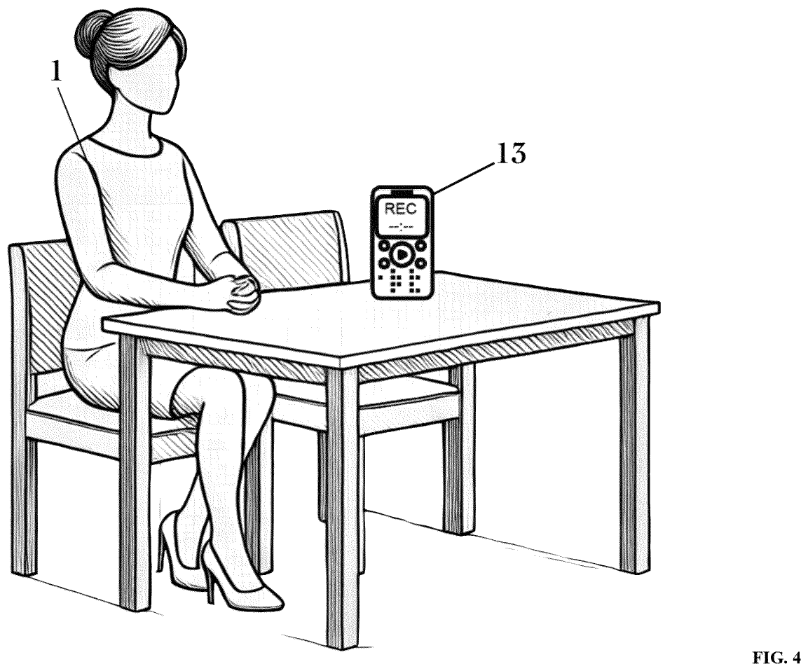

The non-limiting FIG. 4 illustrates person 1 interacting with an audio recording device 13, which may be implemented through various hardware configurations. In one embodiment, the audio recording device 13 may be a smartphone executing a specialized application for capturing and processing audio cues. Alternative implementations may include dedicated voice recorders, smart speakers with recording capabilities, or peripheral devices connected to a computer or smartphone.

The audio recording device 13 may incorporate various audio processing capabilities, including noise reduction, voice enhancement, and audio quality optimization. In some embodiments, the device may support multiple audio input modes, including direct voice recording, selection from pre-recorded audio libraries, or real-time audio synthesis. The device may also support different audio formats and quality levels to optimize storage and playback requirements.

The audio recording functionality may be integrated with various other system components through wireless protocols such as BLUETOOTH, WI-FI, or cellular networks. The recorded audio may be processed through neural networks for feature extraction, emotional content analysis, or quality enhancement. In some embodiments, the system may support multiple audio channels or spatial audio recording to enhance the immersive quality of the audio cues during dream incubation.

Both the scent canister 9 and audio recording device 13 may be configured to communicate with other system components through standardized protocols, enabling seamless integration with the dream incubation system's sleep monitoring and cue delivery mechanisms. The devices may include feedback systems to confirm successful operation and maintain optimal performance levels throughout the dream incubation process.

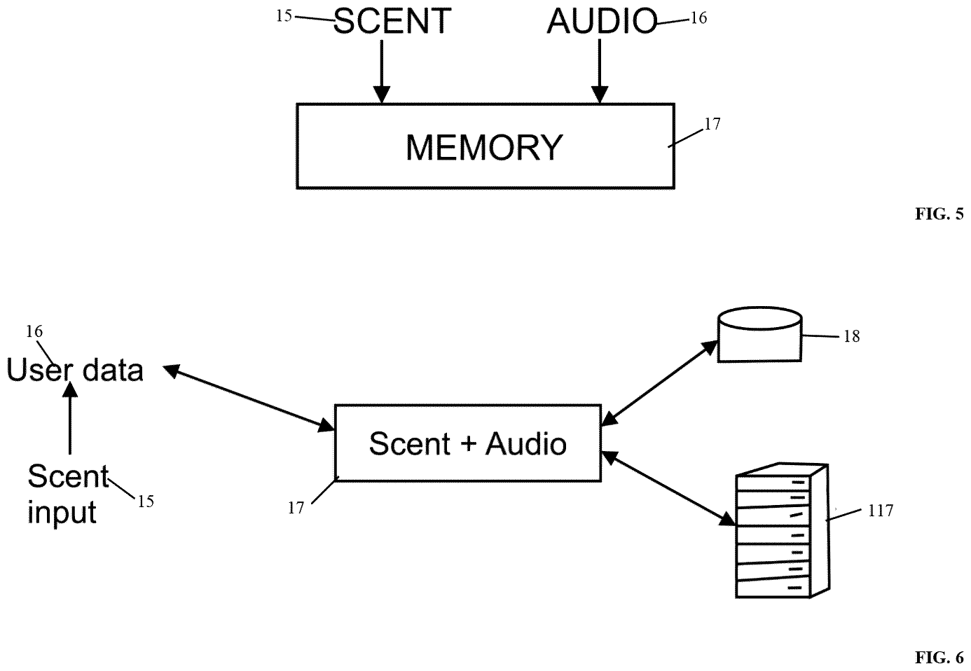

Referring now to FIGS. 5 and 6, exemplary memory creation processes are illustrated according to various aspects of the invention. These figures demonstrate how the sensory experiences described in relation to FIGS. 1-4 are digitally captured, processed, and stored within the system's architecture.

Firstly, the FIG. 5 depicts the exemplary fundamental components of memory creation, wherein a scent cue identifier input 15 and audio cue identifier input 16 are associated to form a memory 17. The scent cue identifier input 15 corresponds to a specific scent or scent combinations available in the scent canister 9 previously described in FIG. 3. For example, when capturing a wedding memory as illustrated in FIG. 1, the scent cue identifier input 15 might reference a floral scent associated with the wedding venue 3, while the audio cue identifier input 16 might contain a recording of wedding vows or significant phrases captured through the audio recording device 13 shown in FIG. 4.

In various embodiments, the scent cue identifier input 15 may comprise a unique digital code, a descriptive name, or a standardized scent classification that maps to specific scent chambers within the scent canister 9. Preferably, the system maintains a digital look-up table or database that correlates these identifiers with physical scent dispensing mechanisms, ensuring accurate scent selection during dream incubation. For instance, when capturing a sports-related memory as shown in FIG. 2, the scent identifier might reference the fresh-cut grass of the venue 6 or other distinctive environmental scents associated with the event 601.

The FIG. 6 further expands upon the embodiment of FIG. 5, illustrating a comprehensive memory creation architecture. The scent cue input 15 and user data input 16 are processed and stored as memory data input 17 within a database 18, which may reside on a server 117. The user data input 16 may encompass various forms of supplementary information about the memorable event, such as photographs, textual descriptions, emotional ratings, or contextual details. For example, in the context of the wedding event 600 shown in FIG. 1, the user data input 16 might include images of the couple 2, details about the venue 3, and personal notes about the emotional significance of the ceremony.

Preferably, the server 17 may implement various data management protocols to maintain the relationships between different memory components. The system may employ various database architectures, such as relational databases for structured data or document-oriented databases for handling diverse media types. In some embodiments, the server 117 may implement distributed storage systems to ensure data redundancy and high availability.

The memory creation process may incorporate multiple validation layers to ensure data integrity and proper association between sensory cues. For instance, when storing a memory related to the football event shown in FIG. 2, the system may validate that the selected scent identifier corresponds to an available scent in the user's scent canister 9, and that the audio recording quality from device 13 meets minimum fidelity requirements for effective dream incubation.

The database 18 may implement various optimization techniques for efficient storage and retrieval of memory data. This includes compression algorithms for audio data, efficient indexing of scent identifiers, and caching mechanisms for frequently accessed memories. The system may also maintain usage statistics and success metrics for different memory-sensory combinations, enabling continuous optimization of the dream incubation process.

Security features may be implemented at various levels, including encryption of sensitive personal data, secure communication protocols between system components, and access control mechanisms for multi-user environments. The server 117 may also implement backup and recovery procedures to protect against data loss and ensure continuity of the dream incubation service.

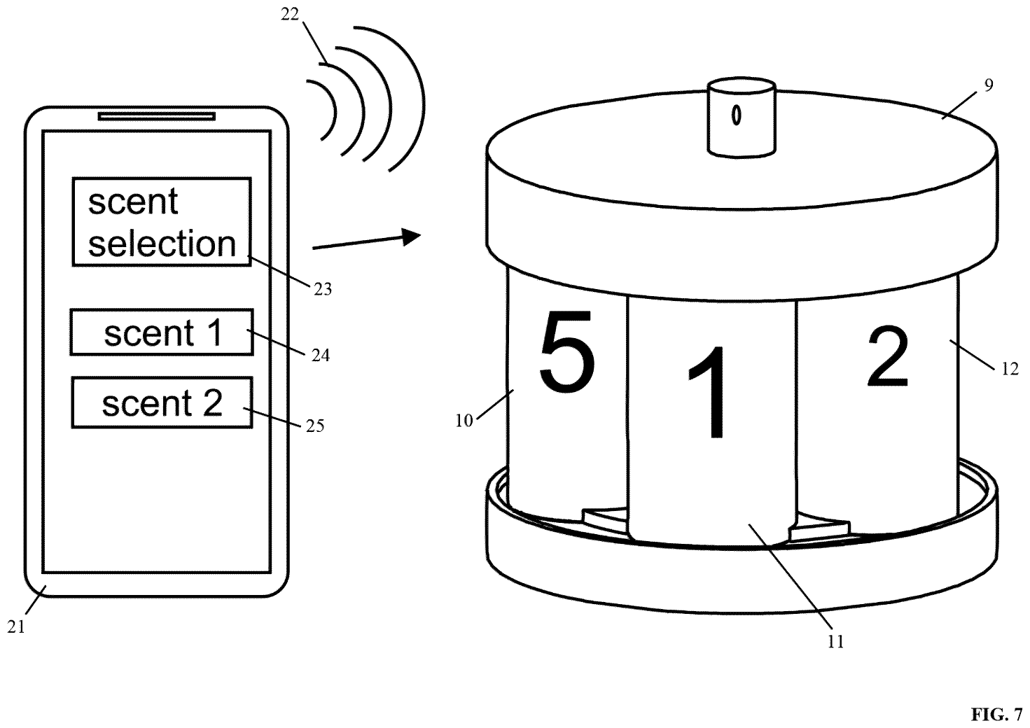

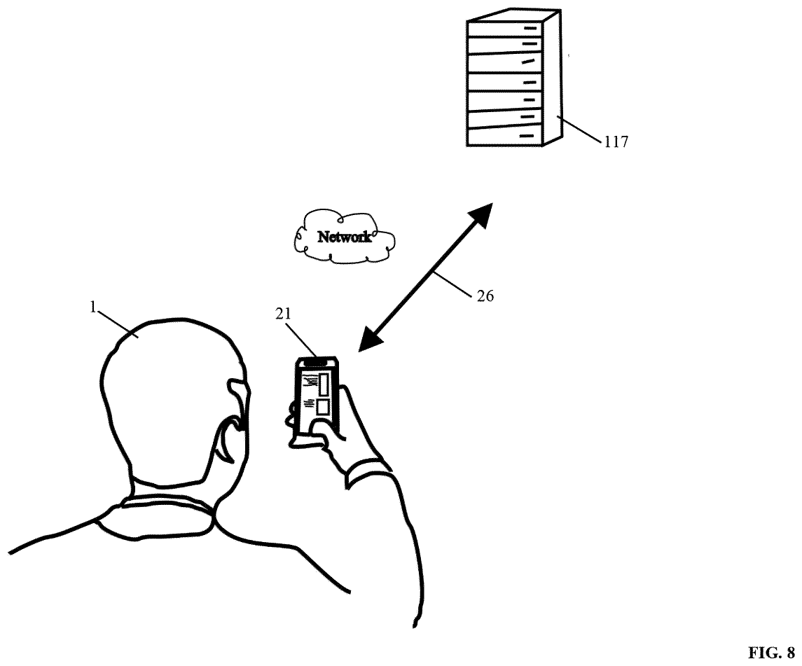

Referring now to FIG. 7, a data coupling architecture between a user device 21 and scent canister 9 is illustrated according to one aspect of the invention. This figure demonstrates the practical implementation of the memory-sensory associations previously described in FIGS. 5 and 6, particularly showing how the stored memory data is translated into physical scent selection and dispensation.

The user device 21 establishes a communication mechanism 22 with the scent canister 9, which may be implemented through various wireless protocols including, but not limited to, BLUETOOTH™ Low Energy (BLE), WI-FI Direct, ZIGBEE™ or proprietary RF protocols. In alternative embodiments, the communication mechanism 22 may be implemented through wired connections such as USB, or through Internet-mediated communication where both the user device 21 and scent canister 9 connect to a common cloud service or any such.

The user device 21 displays a graphical user interface (GUI) element 23 that enables scent selection through interactive elements 24 and 25. For example, element 25 might represent a selection for lavender scent, corresponding to a specific chamber within the canister 9 containing first scent 10, second scent 11, or third scent 12. This interface provides a user-friendly method for accessing the scent-memory associations stored in database 18 (previously described in FIG. 6) and triggering appropriate scent dispensation for memory association or dream incubation purposes.

In various implementations, the GUI elements may present scent options in different ways, such as through descriptive names (e. g., “Wedding Flowers” relating to event 600 in FIG. 1), emotional categories (e. g., “Joyful Sports Moment” relating to event 601 in FIG. 2), or standardized scent classifications. The interface may also display additional memory context stored during the creation process described in FIGS. 5 and 6, helping users select appropriate scent-memory combinations for dream incubation.

Alternative implementations might include voice-controlled selection, gesture-based interfaces, or automated selection based on pre-programmed schedules or sleep patterns. The system may also support more sophisticated scent canister configurations, such as networked arrays of single-scent dispensers, modular scent cartridge systems, or integrated smart home fragrance networks.

The scent canister 9 may implement various feedback mechanisms to confirm successful scent dispensation, such as optical sensors detecting atomization, pressure sensors monitoring scent fluid levels, or electronic flow meters. This feedback can be transmitted back to the user device 21 through communication mechanism 22, enabling the system to maintain accurate records of scent usage and availability.

In some embodiments, the user device 21 may implement local caching of scent-memory associations and control logic, enabling continued operation during temporary network outages. The system may also support multiple user profiles, with appropriate access controls and personalized scent-memory mappings for each user, while maintaining the privacy of individual memory associations as described in the database architecture of FIG. 6.

The communication protocol between user device 21 and scent canister 9 may include error detection and correction mechanisms, automatic retry logic for failed commands, and power management features to optimize battery life in wireless configurations. The system may also support firmware updates for the scent canister 9, enabling future enhancements to scent dispensation capabilities and control algorithms.

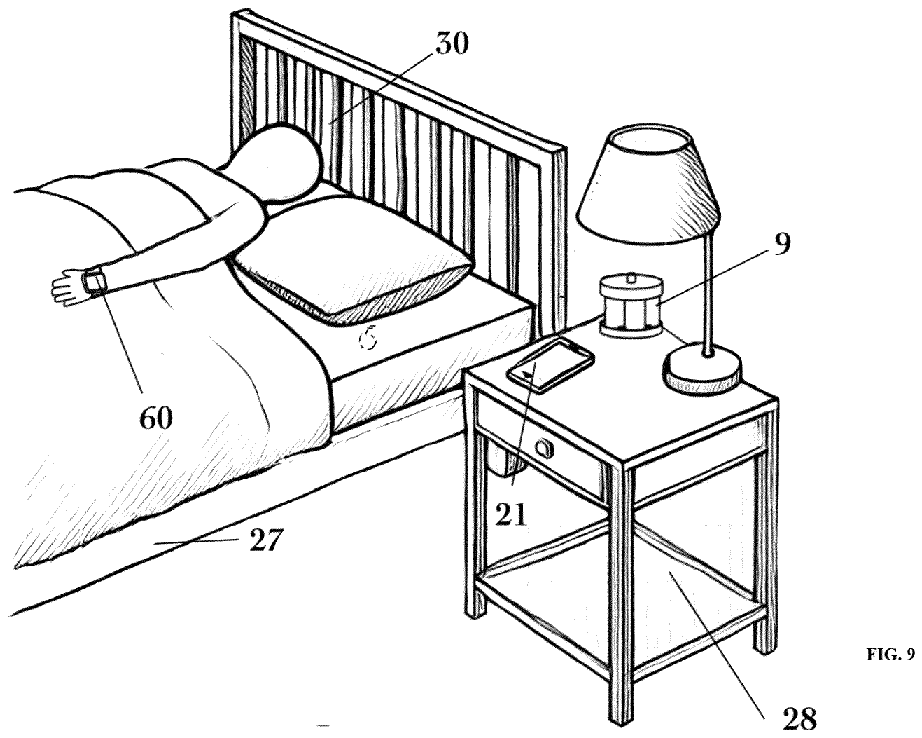

Referring now to FIG. 8, a data communication architecture between a user device 21 and server 117 is illustrated, showing person 1 interacting with the system through a communication network 26. This architecture may enable the cloud-based management and processing of memory-sensory associations previously described in relation to FIGS. 1-7.

The communication network 26 may be implemented through various technologies including cellular networks (4G, 5G), WI-FI™, broadband internet, or other wide-area networking protocols. This network infrastructure facilitates the transmission of memory data, including the scent identifiers and audio cues described in FIGS. 3-4, between the user device 21 and server 117. Alternative implementations may include mesh networks, local area networks, or hybrid networking configurations depending on deployment requirements and available infrastructure.

The server 117, which maintains the memory database 18 described in FIG. 6, may be implemented through various architectures including cloud-based servers, distributed computing networks, or edge computing configurations. In some embodiments, the server infrastructure may be geographically distributed to optimize response times and ensure system reliability. The server may implement load balancing, automatic scaling, and fail-over mechanisms to maintain consistent service quality.

The user device 21, which interfaces with both the server 117 and the scent canister 9 (shown in FIG. 7), serves as the primary control point for memory creation and dream incubation processes. Alternative implementations of the user device may include dedicated hardware controllers, smart home hubs, or specialized dream incubation devices, in addition to conventional smartphones or tablets. The device may implement local processing capabilities to reduce server load and enable offline operation when network connectivity is limited.

The system may support various authentication and security protocols to protect the privacy of person 1's memory data during transmission across communication network 26. This may include end-to-end encryption, secure socket layers (SSL), token-based authentication, or biometric verification methods. The security measures ensure that sensitive memory associations, such as personal events depicted in FIGS. 1-2, remain private and secure.

In alternative embodiments, the system may implement edge computing capabilities on the user device 21, allowing for local processing of time-sensitive operations while maintaining synchronization with server 117 for long-term storage and advanced processing tasks. This hybrid approach can optimize system responsiveness while maintaining the benefits of cloud-based data management and processing.

The communication architecture may also support real-time analytics, enabling the system to adapt and optimize dream incubation parameters based on accumulated user experience data. This feedback loop integrates with the sensory delivery mechanisms described in FIG. 7 to enhance the effectiveness of dream incubation processes over time.

Referring now to FIG. 9, a sleep environment configuration is illustrated according to one aspect of the invention, showing the physical deployment of various system components described in the previous figures. A person 30 is shown sleeping on a bed 27, with a user device 21 and scent canister 9 positioned on a nearby table 28. This arrangement demonstrates the practical implementation of the dream incubation system incorporating the communication architectures described in FIGS. 7 and 8.

The user device 21, which maintains communication with both the scent canister 9 and server 117 (shown in FIG. 8), may be positioned within effective range for both wireless data transmission and audio playback. The placement of scent canister 9 on table 28 is optimized for effective dispersal of the selected scents previously associated with specific memories as described in FIGS. 5 and 6. The positioning may be adjusted based on room size, airflow patterns, and the specific characteristics of the scents being used.

Person 30 is shown wearing a smart watch 60, which serves as one of the bio-sensor devices for sleep state detection. Alternative sensor configurations may include different types of wearable devices, bed-mounted sensors, room-mounted cameras, or contactless radio frequency sensors. The smart watch may integrate with other system components through the communication mechanisms described in FIGS. 7 and 8.

The sleep environment configuration supports the full memory-to-dream translation process, from the initial capture of memorable events (as shown in FIGS. 1-2) through to the coordinated delivery of sensory cues during sleep. The arrangement of components enables the system to monitor sleep states, trigger appropriate sensory cues, and maintain communication with cloud-based services while minimizing disruption to the user's sleep environment.

Alternative implementations may include integrated smart bedroom systems where the scent dispensing and audio playback capabilities are built into room fixtures, or more portable configurations for travel use. The system may also accommodate multiple users in the same space through personalized sensor associations and directed sensory stimulation techniques.

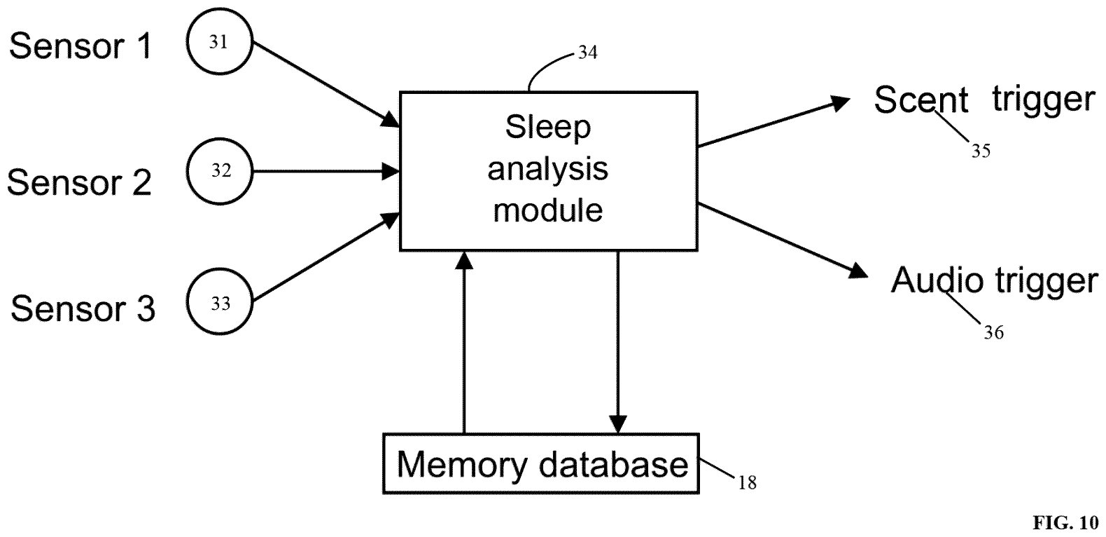

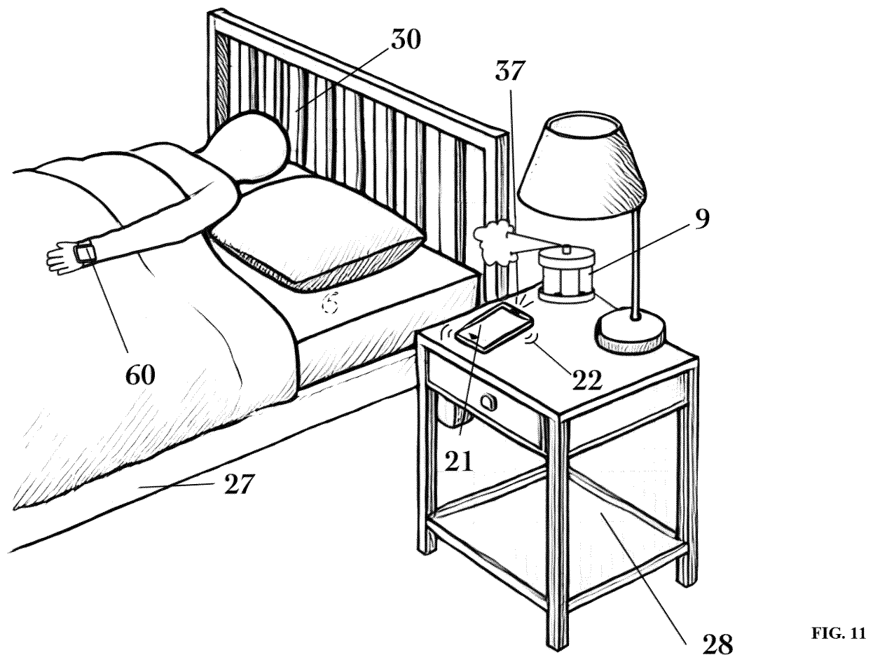

Referring now to FIG. 10, an exemplary flow chart illustrates a sensor-triggered audio and scent cue system according to one aspect of the invention. The system comprises multiple sensor inputs through sensor one 31, sensor two 32, and sensor three 33, which feed into a sleep analysis module 34 that couples with a memories database 18 to trigger appropriate sensory cues through a scent cue trigger 35 and audio trigger 36.

The sensor array (31, 32, 33) may include various types of bio-sensors collecting different physiological and environmental data. Sensor one 31 may be implemented as an accelerometer within a smart watch (as shown in FIG. 9) or smartphone, capturing micro-movements and body position changes at sampling rates between 50-200 Hz. The accelerometer data provides crucial information about sleep onset patterns and body movement frequencies characteristic of different sleep stages.

Sensor two 32 may comprise heart rate monitoring capabilities, implemented through optical sensors in wearable devices or through radio frequency-based contactless monitoring systems. The heart rate data includes both instantaneous heart rate and heart rate variability metrics, sampled at appropriate intervals to detect the subtle cardiovascular changes that accompany transitions between sleep stages.

Sensor three 33 may be implemented as a microphone array, either in the user device 21 (shown in FIG. 9) or as dedicated sleep monitoring equipment. The audio sensors capture breathing patterns, ambient noise levels, and other acoustic signatures relevant to sleep stage classification, with sampling rates and filtering optimized for sleep monitoring applications.

The sleep analysis module 34 implements a sophisticated neural network architecture for processing the multi-modal sensor data. In one embodiment, the module employs separate neural networks for each sensor type, with convolutional layers processing time-series data from the accelerometer and heart rate sensors, while recurrent neural networks handle sequential audio data. These separate networks feed into a fusion layer that combines the processed sensor data to make sleep stage classifications.

Alternative implementations of the sleep analysis module 34 may utilize end-to-end neural network architectures, processing all sensor inputs through a unified network structure. The module may implement either deep Q-learning or policy gradient methods for optimizing sensory cue timing, with the reward function weighing both bio sensor data and subsequent user feedback equally.

The sleep analysis module 34 interfaces with the memories database 18, which contains the stored memory associations created through the processes described in FIGS. 5 and 6. Upon detecting the N1 NREM sleep stage, the module triggers appropriate sensory responses through the scent cue trigger 35 and interfaces with the audio trigger 36 for coordinated audio playback.

The scent cue trigger 35 communicates with the scent dispensing device 9 (shown in FIG. 9) through the communication mechanisms detailed in FIG. 7, activating the appropriate scent chamber based on the selected memory's scent identifier. The trigger implementation includes error checking and confirmation mechanisms to ensure reliable scent delivery.

The scent trigger 36 manages the audio cue playback, coordinating with the user device 21 or other audio output devices to deliver the associated audio stimuli. The timing between scent and audio cue delivery may be adjusted based on learned optimization parameters stored in the sleep analysis module 34.

Preferably, the entire flow process implements error handling and feedback collection mechanisms. The system continuously monitors sensor data quality, adjusts for environmental variations, and maintains synchronization between different system components. Alternative implementations may include additional sensor types, such as temperature sensors, galvanic skin response monitors, or EEG devices, with the sleep analysis module 34 architecture adapted to process these additional inputs.

Referring now to FIG. 11, a comprehensive dream incubation implementation is illustrated, showing the coordinated delivery of sensory cues to person 30 sleeping on bed 27. This figure demonstrates the practical culmination of the sensor processing and trigger mechanisms described in FIG. 10, resulting in the synchronized delivery of audio cue 37 from user device 21 and scent cue 38 from scent canister 9.

The smart watch 60 worn by the sleeping person continuously monitors physiological parameters, transmitting data through the previously described sensor pathways. Upon positive detection of the target sleep phase, the system initiates a carefully orchestrated sequence of sensory stimulation. The communication mechanism 22 between the user device 21 and scent canister 9, positioned on table 28, enables precise temporal coordination of the sensory cues.

The audio cue 37 may be implemented through various output configurations beyond the user device 21's speakers. Alternative audio delivery systems may include directional speakers, bone conduction devices, or smart pillow speakers that minimize disturbance to other room occupants while maintaining effective audio delivery to the sleeping person. The system may modulate audio volume based on ambient noise levels detected through the device microphones, ensuring optimal audibility without disrupting sleep.

The scent cue 38 delivery may implement dynamic adjustment mechanisms based on environmental factors. The system may incorporate airflow sensors to detect room ventilation patterns and adjust dispersion timing accordingly. Machine learning algorithms can optimize scent intensity and duration based on factors such as room size, humidity, and historical effectiveness data.

Feedback collection during the dream incubation process may occur through multiple channels. Primary physiological feedback comes through the smart watch 60, which monitors immediate bodily responses to the sensory stimuli. The system tracks parameters such as heart rate variability changes, motion patterns, and skin conductance responses to assess the effectiveness of the cue delivery.

Implementation variations may include closed-loop feedback systems that make real-time adjustments to cue delivery based on physiological responses. For example, if initial physiological markers indicate insufficient response to the sensory cues, the system may adjust the intensity or duration of subsequent cues within pre-defined safety parameters.

The user device 21 may implement edge computing capabilities to process immediate feedback and make time-critical adjustments while maintaining synchronization with cloud-based services for longer-term optimization. This hybrid processing approach enables responsive adaptation to individual sleep patterns while contributing to the system's learning and optimization processes.

Success validation incorporates both objective and subjective measures. The system logs quantitative data about sleep quality metrics, physiological responses, and environmental conditions during cue delivery. Upon waking, the user may provide qualitative feedback through the user device 21 interface, rating dream vividness, emotional resonance, and memory correlation. This multi-modal feedback approach enables continuous refinement of the dream incubation parameters.

The implementation may support fail-safe mechanisms and error recovery procedures. If communication is lost between components, the system may maintain core functionality through local control loops while attempting to re-establish full connectivity. Backup power systems may ensure consistent operation of critical components throughout the sleep period.

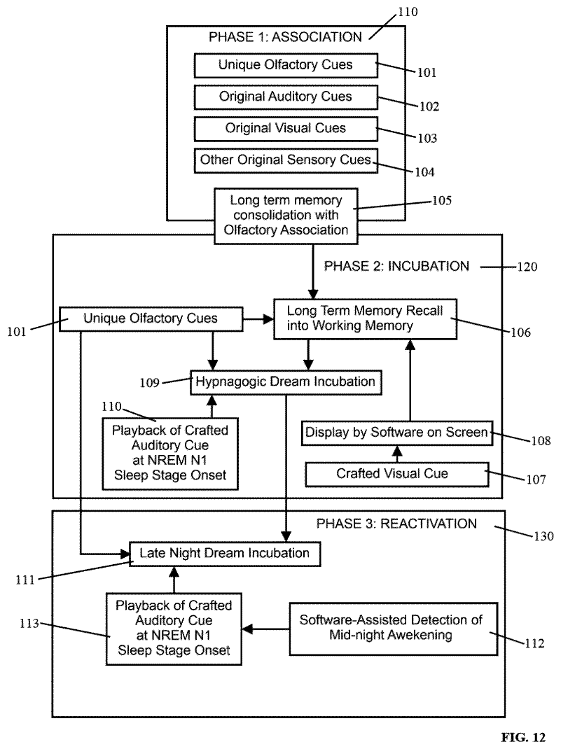

Referring now to FIG. 12, a flow diagram illustrates three distinct phases of memory-to-dream translation: an association phase 110, an incubation phase 120, and an optional reactivation phase 130. The diagram preferably represents the temporal progression and interaction of various sensory inputs and cognitive processes throughout the dream incubation methodology.

In the exemplary association phase 110, the system may preferably facilitate the creation of strong memory anchors through multi-sensory integration. A unique olfactory cue 101 may be systematically paired with environmental sensory inputs, which may include original auditory cues 102, original visual cues 103, and other original sensory cues 104. The olfactory cue 101 may preferably be provided through the scent canister 9 previously described in relation to FIGS. 3 and 7, delivering precisely controlled scent stimuli that serve as primary memory anchors.

The environmental cues 102, 103, and 104 may correspond to naturally occurring sensory experiences associated with memorable events, such as those described in FIGS. 1 and 2. For instance, during a wedding event 600, the auditory cues 102 may include ceremonial music, while visual cues 103 may comprise the wedding setting and participants. The system may preferably allow for variable presence and intensity of these environmental cues, recognizing that not all memories will incorporate equal sensory richness.

The incubation phase 120 may be initiated prior to intended sleep periods, preferably utilizing the sleep environment configuration detailed in FIG. 9. This phase may begin with the controlled release of the original olfactory cue 101, triggering memory recall 106 into the user's working memory system. The process may be optionally enhanced through the presentation of crafted visual cues 107 via a smartphone or similar device 108, which may preferably display contextually relevant imagery or text associated with the target memory.

The system may preferably time the transition to hypnagogic dream incubation 109 using the sleep phase detection mechanisms detailed in FIG. 10. Upon detecting N1 NREM sleep stage onset through the described sensor array, the system may initiate playback of crafted auditory cues 110. These cues may be delivered through various means, including but not limited to pre-recorded user vocalizations, live partner delivery, or software-controlled audio systems as described in relation to FIG. 11.

The optional reactivation phase 130 may implement additional dream reinforcement mechanisms during natural sleep cycle variations. A software subsystem 112 may preferably utilize the sensor data processing capabilities described in FIG. 10 to detect mid-night awakenings 113. Upon such detection, the system may initiate late-night dream incubation 111 through carefully timed delivery of crafted auditory cues. The system may optionally trigger additional releases of the original olfactory cue 101 through the communication mechanisms detailed in FIG. 7, potentially enhancing dream immersion and recall probability.

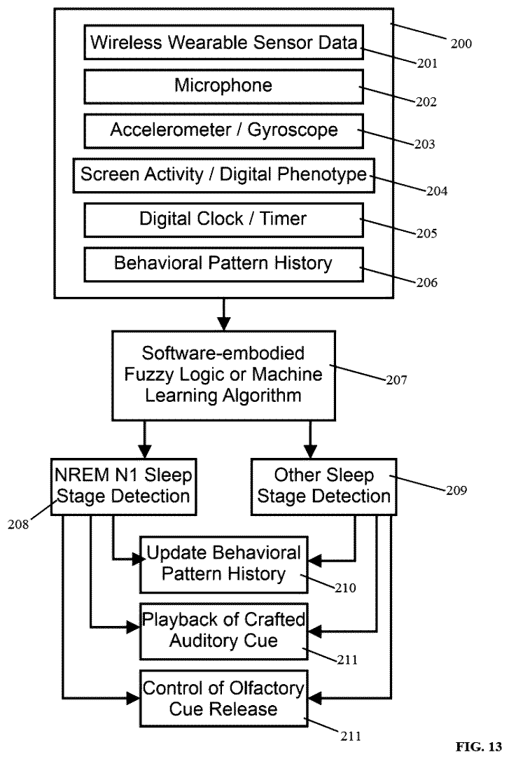

Referring now to FIG. 13, an exemplary non-limiting machine learning implementation for sleep phase detection and sensory cue optimization may preferably be described. The system may receive a plurality of bio-sensor data inputs 200, which may preferably be processed through multiple specialized neural network architectures to enable precise sleep stage identification and optimal cue delivery timing.

The bio-sensor data inputs 200 may preferably comprise multiple data streams that may be systematically analyzed for sleep stage determination. The accelerometer data may preferably be sampled at 50-200 Hz to capture micro-movements characteristic of sleep phase transitions. In a first exemplary aspect, hypnic jerks indicating N1 NREM sleep onset may manifest as sudden acceleration spikes typically ranging from 0.1 to 0.5 g, which may preferably be sampled at 100 Hz to optimize detection while maintaining efficient power usage.

Heart rate variability data may preferably be captured through photoplethysmography (PPG) sensors in wearable devices, or alternatively through radio frequency-based contactless monitoring. In a second exemplary aspect, the system may analyze RMSSD (Root Mean Square of Successive Differences) and pNN50 metrics over rolling 5-minute windows with 1-minute overlaps. The transition to N1 sleep may be characterized by RMSSD values increasing from 20-40 ms during wakefulness to 40-60 ms during early sleep stages.

Acoustic data may preferably be captured through smartphone microphones, sampled at 16 kHz with appropriate noise filtering. In a third exemplary aspect, breathing pattern analysis may identify sleep onset through frequency transitions from 12-16 breaths per minute during wakefulness to 9-14 breaths per minute during N1 sleep, with analysis preferably focused on the 100-500 Hz frequency range.

For example, a typical sleep onset sequence may demonstrate the following patterns:

Initial wake state may preferably be characterized by: Accelerometer readings showing irregular movements exceeding 0.3 g; Heart rate measurements between 65-75 BPM with RMSSD below 30 ms; and Breathing patterns showing irregularity at 14-16 breaths per minute

Drowsiness transition may preferably exhibit: Movement amplitude reduction to below 0.2 g; Heart rate decrease to 60-70 BPM with increasing RMSSD; Breathing pattern regularization at 12-14 breaths per minute

N1 NREM onset may preferably display: Occasional hypnic jerks registered as 0.3-0.5 g acceleration spikes; Stable heart rate at 55-65 BPM with RMSSD exceeding 40 ms; and Regular breathing patterns at 9-11 breaths per minute

The machine learning architecture may preferably implement a hybrid model combining supervised and reinforcement learning approaches. The neural network may preferably be trained on labeled polysomnography data, where ground truth sleep stages have been identified by sleep experts. The training dataset may preferably include diverse subject populations and varying environmental conditions to ensure robust generalization.

The neural network architecture may preferably employ a multi-stream approach implementing separate processing pathways for each sensor type. In a fourth exemplary aspect, the accelerometer data stream may be processed through 1D convolutional layers with varying kernel sizes (preferably 32, 64, and 128 samples) to capture movement patterns at different temporal scales. The system may preferably implement stride lengths of 8 samples with appropriate padding to maintain temporal resolution.

Heart rate variability data may preferably be analyzed through a combination of convolutional and recurrent layers. In a fifth exemplary aspect, the system may employ a two-stage processing pipeline where initial convolutional layers extract beat-to-beat interval features, followed by LSTM layers with 128 hidden units processing sequences of 300 heartbeats to capture longer-term patterns.

The acoustic data processing stream may preferably implement a specialized architecture optimized for breathing sound analysis. In a sixth exemplary aspect, the system may employ mel-frequency cepstral coefficients (MFCCs) computed over 25 ms windows with 10 ms overlap, followed by convolutional layers for feature extraction and bidirectional LSTM layers with 256 hidden units for temporal pattern recognition.

Additional sensor inputs may preferably include: Temperature variations: typically showing a 0.5-1.5° C. drop during sleep onset; Skin conductance: demonstrating characteristic reduction patterns; and Body position changes: showing reduced frequency and amplitude

The reinforcement learning component may preferably optimize sensory cue timing through either deep Q-learning or policy gradient methods. The state space may comprise processed sensor data and historical response patterns, while the action space may include decisions about cue timing and intensity. In a seventh exemplary aspect, the reward function may incorporate: Immediate physiological responses (40% weighting); Subsequent dream report quality metrics (40% weighting); and User satisfaction ratings (20% weighting)

The system may preferably implement continuous model adaptation through federated learning approaches, enabling personalization while maintaining user privacy. Error handling may include confidence thresholds for sleep stage detection, requiring minimum confidence levels of 85% before triggering sensory cues.

In alternative aspects, the system may implement an alternative sleep stage detection methodology utilizing computer vision analysis through a camera-based monitoring approach. In this embodiment, the system may employ infrared or low-light cameras positioned to capture subtle physiological indicators during sleep, while maintaining user privacy through on-device processing and abstracted data representation.

The video processing pipeline may preferably utilize deep convolutional neural networks optimized for detecting micro-movements and physiological signals. The system may implement remote photoplethysmography (rPPG) techniques to extract heart rate variability data from subtle skin color variations, which may be captured through specialized image processing at 30-60 frames per second. This non-contact approach may preferably detect pulse rate changes characteristic of sleep stage transitions, with typical accuracy within 2-3 beats per minute compared to contact sensors.

Motion analysis may preferably be performed through optical flow computation and temporal difference imaging, enabling the detection of respiratory patterns and body position changes. The system may track breathing rates through chest and abdominal movement analysis, with sub-pixel motion detection algorithms capable of identifying breathing rates in the range of 9-16 breaths per minute. Additionally, rapid eye movement detection during different sleep stages may be accomplished through specialized eye region monitoring when the user's face is visible to the camera.

The neural network architecture for video processing may preferably implement a two-stream approach, combining spatial and temporal information processing. The spatial stream may analyze individual frames for posture and position information, while the temporal stream may process sequences of frames to detect movement patterns and physiological rhythms. These streams may converge in a fusion layer that may preferably implement attention mechanisms to focus on the most relevant features for sleep stage classification.

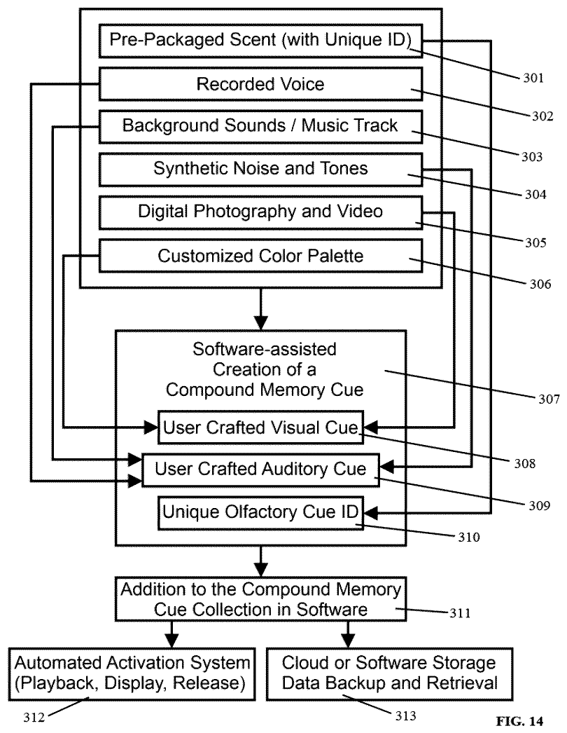

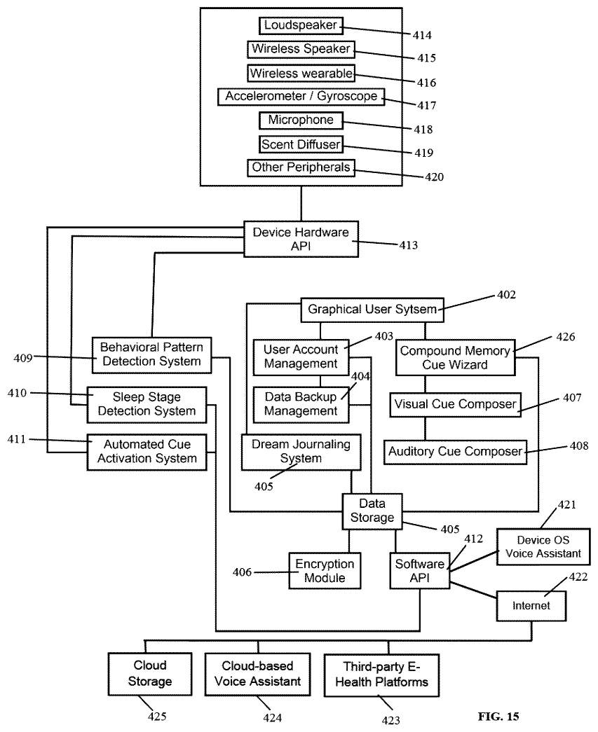

Referring now to FIG. 14, a comprehensive diagram illustrates the creation and management of crafted sensory cues within the software embodiment of the invention. The system may preferably incorporate multiple input pathways for creating compound memory associations, building upon the sensory integration mechanisms previously described in relation to FIGS. 5 and 6.

The system may preferably begin with a series of input components 301 through 306, which may enable sophisticated multi-modal memory cue creation. The fundamental input 301 may comprise a unique identifier associated with pre-packaged scents, corresponding to the scent selection mechanisms detailed in FIG. 7. This olfactory component may preferably serve as the primary anchor for memory association, maintaining consistency with the scent dispensation system described in previous figures.

Audio components may preferably be processed through inputs 302, 303, and 304, enabling a rich auditory experience. The system may capture voice recordings through input 302, which may utilize the audio recording capabilities described in FIG. 4. Background audio elements through input 303 may preferably include ambient sounds or music, while input 304 may enable the integration of synthesized tones optimized for sleep-stage specific playback as detailed in FIG. 11.

Visual components may be processed through inputs 305 and 306, though these may remain optional within the dream incubation process. Digital imagery through input 305 may preferably support various formats including still photographs and video sequences, while input 306 may enable the specification of color palettes that may enhance memory association during the wake state.

The software integration process 307 may preferably implement sophisticated algorithms for combining these sensory inputs into coherent compound memory cues. The system may utilize the unique olfactory cue ID 308 as a primary reference point, around which user-crafted auditory cues 309 and optional visual cues 310 may be organized. This integration may preferably maintain temporal synchronization between components while optimizing each element for dream incubation effectiveness.

The resulting compound memory cues may preferably be managed through a collection system 311, which may implement the database architecture previously described in relation to FIG. 6. Each compound cue may maintain a strict one-to-one relationship with its associated olfactory identifier, ensuring consistent sensory experiences during dream incubation.

The automated activation system 312 may preferably coordinate the delivery of these compound cues, implementing the sleep stage detection and timing optimization mechanisms detailed in FIG. 13. This system may preferably adapt cue delivery parameters based on real-time physiological feedback and historical effectiveness data.

Cloud integration 313 may preferably provide secure storage and synchronization capabilities, enabling seamless access across multiple devices while maintaining data privacy. This may implement the communication architecture described in FIG. 8, with appropriate encryption and access control mechanisms.

Referring now to FIG. 15, a system architecture illustrates the integration of memory-to-dream translation components in accordance with the invention. The architecture may preferably be centered around a processing system 401 that implements the neural network-driven dream incubation functionality through multiple integrated modules.

A user interface 402 may preferably provide memory creation and sensory association capabilities as described in FIG. 5, while account management 403 and data preservation 404 modules may maintain user-specific configurations within the secure storage system 405.

The memory-sensory association module 426 may preferably implement the multi-modal integration mechanisms detailed in FIG. 14, incorporating both visual processing 407 and audio processing 408 components. These elements may preferably communicate with bio-sensors through the hardware interface 413, enabling the creation and management of memory anchors as described in FIG. 12.

The system may preferably implement physiological pattern recognition 409 and sleep phase detection 410 utilizing the neural network architectures detailed in FIG. 13. These components may process inputs from the wearable device 416, motion sensors 417, and acoustic sensors 418, implementing the sensor fusion and sleep classification mechanisms previously described in FIG. 10.

The sensory cue coordination system 411 may preferably manage stimulus delivery through audio output devices 414, 415, and the scent dispensing device 419, maintaining the precise temporal synchronization detailed in FIG. 11. The system may interface with these components through the hardware interface 413, implementing the communication protocols described in relation to FIG. 8.

A communication interface 412 may preferably enable integration with external systems through network connectivity 422, facilitating data exchange with health monitoring platforms 423, voice control systems 424, and remote storage 425. This connectivity may implement the secure communication architecture described in FIG. 8, ensuring data privacy and system reliability.

The dream experience recording system 405 may preferably capture user feedback and dream reports, providing essential data for the reinforcement learning mechanisms detailed in FIG. 13. The secure storage system 405 may implement encryption protocols through security encryption module 406, maintaining data integrity across all system components.

The architecture may preferably support additional bio-sensors 420 through standardized interfaces, enabling system expandability while maintaining consistent performance across various hardware configurations. This modular approach may align with the sensor integration capabilities described in FIG. 10, ensuring robust operation across different deployment scenarios.

In some aspects, the system may determine sleep stage transitions through additional physiological markers, for example, including skin temperature variations ranging from 0.3-1.0° C. during sleep onset, galvanic skin response changes showing 10-30% reduction in conductance, and alterations in muscle tension detected through EMG readings showing 40-60% amplitude reduction during N1 NREM onset.

In certain embodiments, the scent dispensing device may implement multiple dispensation mechanisms including piezoelectric atomization for example, operating at 100-200 kHz, thermal vaporization maintaining precise temperature control for example, between 30-60° C., or ultrasonic nebulization at 1-2 MHz. The selection of dispensation mechanism may depend on the specific chemical properties of the scents being utilized.

In some implementations, the neural network architecture may employ attention mechanisms with multiple attention heads, typically 4-8 heads per layer, enabling the system to focus on different temporal aspects of the bio-sensor data simultaneously. The attention weights may be dynamically adjusted based on the quality of sleep stage detection, with higher weights assigned to more reliable sensor inputs.

In particular embodiments, the audio processing system may implement adaptive noise cancellation using reference microphones to isolate breathing sounds for example, with filter coefficients updated at 100 Hz to maintain optimal noise rejection. The system may employ spatial filtering techniques when multiple microphones are available, improving the signal-to-noise ratio by 15-20 dB.

In some configurations, the reinforcement learning system may implement a hierarchical reward structure where immediate physiological responses for example, contribute 40% of the reward signal, dream report quality metrics provide 40%, and user satisfaction ratings account for 20%. The reward function may be updated every 100 training episodes to optimize the balance between these components.

In certain aspects, the system may employ transfer learning techniques to adapt pre-trained neural networks to individual users while maintaining generalization capabilities. The adaptation process may, for example, require 5-10 nights of user-specific data to achieve optimal performance, with continuous fine-tuning thereafter.

In some embodiments, the memory association process may implement a temporal alignment algorithm ensuring synchronization between olfactory and auditory cues with maximum allowable temporal deviation of, for example, 100 ms. The system may maintain this synchronization through a distributed clock system with network time protocol synchronization.

In particular implementations, the system may employ federated learning techniques allowing model improvements without centralizing user data. Local model updates may, for example, be computed every 3-5 nights of use, with aggregated model updates distributed weekly while maintaining user privacy.

In some aspects, the system may implement different dream incubation protocols based on user chronotype, adjusting timing parameters by, for example, ±30-45 minutes to align with individual circadian rhythms. The chronotype assessment may be performed through analysis of movement patterns and physiological data over, for example, 5-7 days of baseline recording.

### Benefits of the Invention

The present invention provides significant advantages in the field of dream incubation and memory processing. By implementing precise coordination between olfactory and auditory stimuli during specific sleep phases, the system enables reliable translation of selected memories into dream experiences. The neural network-driven sleep phase detection offers superior accuracy compared to traditional sleep monitoring approaches, while the multi-modal sensory integration creates stronger memory anchors for dream incubation. The system's adaptive learning capabilities allow for continuous optimization of cue timing and intensity, improving success rates over time. The invention's non-invasive nature and integration with common consumer devices such as smartphones and wearables makes it practical for widespread adoption. Additionally, the system's potential applications extend beyond entertainment into therapeutic contexts, potentially aiding in trauma processing, creative problem-solving, and memory consolidation. The secure, privacy-focused architecture ensures user data protection while enabling valuable dream experience documentation.

### INDUSTRIAL APPLICATION

This invention finds significant industrial application in the fields of sleep science, mental health therapy, and cognitive enhancement. The system can be commercially deployed in sleep clinics and therapeutic settings for treating sleep disorders, PTSD, and anxiety-related conditions. Its application extends to creative industries for enhancing problem-solving capabilities through directed dream experiences. The technology can be integrated into existing smart home systems and health-care devices, providing opportunities for consumer electronics manufacturers. The machine learning components enable continuous improvement of dream incubation success rates, making the system valuable for both clinical research and commercial sleep technology applications.

What is claimed is:

1. A system for inducing memory-based dreams, comprising:
a scent dispensing device comprising at least one scent chamber, each chamber containing a scent and associated with a unique scent identifier;
at least one bio-sensor device configured to detect sleep state parameters of a user;
a memory storing a plurality of user memories, each memory associated with at least one scent identifier and at least one audio cue;
a processor in communication with the scent dispensing device, the at least one bio-sensor device, and the memory, the processor configured to:
receive sleep state parameters from the at least one bio-sensor device;
determine, using a neural network trained on sleep pattern data, an occurrence of an N1 NREM sleep stage based on the received sleep state parameters;
select a stored memory from the plurality of user memories;
trigger, in response to determining the N1 NREM sleep stage, release of a scent from the scent dispensing device corresponding to the scent identifier associated with the selected memory; and
initiate playback of the audio cue associated with the selected memory in temporal proximity to the scent release.

2. The system of claim 1, wherein the processor is further configured to update, based on subsequent bio-sensor data and user feedback, parameters for future scent release timing and audio cue playback.

3. The system of claim 2, wherein updating the parameters comprises applying a reinforcement learning algorithm that treats the bio-sensor data and user feedback as equal weights in a reward function.

4. The system of claim 1, wherein the at least one bio-sensor device comprises one or more of: a wearable device, a smartphone accelerometer, a smartphone microphone, and a smartphone camera.

5. The system of claim 1, wherein the neural network comprises separate neural networks for processing data from different types of bio-sensors, the separate neural networks feeding into a fusion layer.

6. The system of claim 1, wherein the processor communicates with the scent dispensing device using at least one of: Bluetooth, Wi-Fi, and Internet protocols.

7. The system of claim 1, wherein the neural network comprises a hybrid architecture combining convolutional layers for processing time-series data with bidirectional LSTM layers for processing sequential data.

8. A method for inducing memory-based dreams, comprising:
receiving sleep state parameters from at least one bio-sensor device configured to detect sleep state parameters of a user;
determining, using a neural network trained on sleep pattern data, an occurrence of an N1 NREM sleep stage based on the received sleep state parameters;
selecting a stored memory from a plurality of user memories stored in a memory, each memory associated with at least one scent identifier and at least one audio cue;
triggering, in response to determining the N1 NREM sleep stage, release of a scent from a scent dispensing device corresponding to the scent identifier associated with the selected memory; and
initiating playback of the audio cue associated with the selected memory in temporal proximity to the scent release.

9. The method of claim 8, further comprising updating, based on subsequent bio-sensor data and user feedback, parameters for future scent release timing and audio cue playback.

10. The method of claim 9, wherein updating the parameters comprises applying a reinforcement learning algorithm that treats the bio-sensor data and user feedback as equal weights in a reward function.

11. The method of claim 8, wherein the neural network comprises a hybrid architecture combining convolutional layers for processing time-series data with bidirectional LSTM layers for processing sequential data.

12. The method of claim 8, further comprising processing bio-sensor data using separate neural networks for different types of bio-sensors and combining outputs using a fusion layer.

13. The method of claim 8, further comprising creating a new memory association by:
receiving a selection of a scent identifier corresponding to a scent in the scent dispensing device;
recording an audio cue; and
storing the scent identifier and audio cue as a new memory in the plurality of user memories.

14. The method of claim 8, wherein determining the N1 NREM sleep stage comprises processing data from one or more of: a wearable device, a smartphone accelerometer, a smartphone microphone, and a smartphone camera.

15. A non-transitory computer-readable medium storing instructions that, when executed by a processor, cause the processor to perform operations comprising:
receiving sleep state parameters from at least one bio-sensor device configured to detect sleep state parameters of a user;
determining, using a neural network trained on sleep pattern data, an occurrence of an N1 NREM sleep stage based on the received sleep state parameters;
selecting a stored memory from a plurality of user memories stored in a memory, each memory associated with at least one scent identifier and at least one audio cue;
triggering, in response to determining the N1 NREM sleep stage, release of a scent from a scent dispensing device corresponding to the scent identifier associated with the selected memory; and
initiating playback of the audio cue associated with the selected memory in temporal proximity to the scent release.

16. The non-transitory computer-readable medium of claim 15, wherein the operations further comprise updating, based on subsequent bio-sensor data and user feedback, parameters for future scent release timing and audio cue playback.

17. The non-transitory computer-readable medium of claim 16, wherein updating the parameters comprises applying a reinforcement learning algorithm that treats the bio-sensor data and user feedback as equal weights in a reward function.

18. The non-transitory computer-readable medium of claim 15, wherein the neural network comprises separate neural networks for processing data from different types of bio-sensors, the separate neural networks feeding into a fusion layer.

19. The non-transitory computer-readable medium of claim 15, wherein the operations further comprise creating a new memory association by:
receiving a selection of a scent identifier corresponding to a scent in the scent dispensing device;
recording an audio cue; and
storing the scent identifier and audio cue as a new memory in the plurality of user memories.

20. The non-transitory computer-readable medium of claim 15, wherein the neural network comprises a hybrid architecture combining convolutional layers for processing time-series data with bidirectional LSTM layers for processing sequential data.
